(19) United States
(12) Patent Application Publication
(10) Pub. No.: US 2002/0002318 A1
(43) Pub. Date: Jan. 3, 2002

(54) PROCESS FOR CONVERSION OF WELL GAS BY DISPROPORATIONATION TO SALEABLE PRODUCTS

(76) Inventors: DENNIS J. O'REAR, PETALUMA, CA (US); DONALD H. MOHR JR., ORINDA, CA (US); CONG-YAN CHEN, RICHMOND, VA (US); PETER J. WHITE, PLEASANT HILL, CA (US)

Correspondence Address: CHEVRON CORPORATION LAW DEPARTMENT, PATENT DIVISION, PO BOX 6006, SAN RAMON, CA, 94583-0806, US

(21) Appl. No.: 09/388,503

(22) Filed: Sep. 2, 1999

Related U.S. Application Data
(63) Continuation-in-part of application No. 09/330,886, filed on Jun. 11, 1999, now abandoned.

(51) Int. Cl. C07C 6/08

(52) U.S. Cl. 585/708

(57) ABSTRACT

A process for partially converting well gas to saleable products on site by disproportionation of the alkanes in the well gas into higher and lower molecular weight alkanes.

### CROSS REFERENCE TO RELATED APPLICATIONS

This application is a continuation-in-part of U.S. patent application Ser. No. 09/330,886 filed Jun. 11, 1999, the entire contents of which are herein incorporated by reference.

### BACKGROUND OF THE INVENTION

1. Field of the Invention

This invention relates to the partial conversion of well gas by disproportionation to saleable products by converting some of the alkanes in the well gas to syncrude and marketable gaseous fractions. The process of the invention is particularly useful in disposing of non-marketable gas in remote locations.

2. Description of the Related Art

The petroleum industry is concerned with the best possible extraction of monetary value from crude oil and/or natural gas trapped in subterranean geological structures known as reservoirs. A well penetrating a reservoir allows hydrocarbons in the reservoir to be transported to the surface. In many cases the hydrocarbons flowing to the surface comprise a mixture of chemicals with different boiling points, and before they can be transported to market they must be separated into those fractions which are stable liquids at atmospheric pressure and temperature and those fractions which are not. In many instances these later gaseous fractions contain a mixture of propane and butane, often referred to as liquid petroleum gas or LPG, which is not of sufficient commercial value to justify its export. Commonly these fractions which are of marginal commercial value, which include LPG, may be consumed to satisfy local need for fuel or are disposed of by flaring or are reinjected into the reservoir. In each instance, much of the potential value of the LPG or other non-marketable gases is lost. In addition, the disposition of the non-marketable gases represent an operating expense.

The options for the use or disposal of the unmarketable gas, such as LPG, in remote locations are limited. Conversion to syncrude by means of existing technology is complex and expensive and cannot be justified from an economic perspective. Flaring also may not be satisfactory for environmental reasons. Reinjection of the gas as a means of disposal may be an available option, but reinjection will result in the loss of potentially valuable products. This problem would be avoided if the technology were available to economically convert the unsaleable gas to syncrude.

Well gas, which is recovered from an oil and gas well, in this disclosure refers to the non-condensed products from the well that remain after fractionation to produce vapor-pressure specification crude oil. Following recovery of the saleable fractions of the well gas, the remaining lighter alkanes, which usually consists of propane and butane and possibly methane, ethane, and pentane, are of less economic value. This gaseous fraction is referred to in this disclosure as light hydrocarbon waste gas. As used in this disclosure the term well gas also included natural gas, especially what is generally referred to as “wet natural gas”. “Wet natural gas” refers natural gas which contains a significant amount of C₃ plus alkanes.

The term “syncrude”, as used in this disclosure refers to those alkanes recovered from the normally unsaleable gas after their conversion by the invention described in this specification to fractions which may be blended with the crude oil product or shipped separately. Syncrude usually refers to a C₅ plus fraction, i.e., a mixture containing molecules mostly having at least five carbon atoms. Depending on the market, the C₅ fraction, i.e., pentane fraction, is sometimes considered to be part of the LPG fraction. For the purposes of this disclosure the C₅ fraction may be included in either the LPG or the syncrude fraction depending on the market opportunities available. In some instances it may be desirable to separately export the pentane as a product apart from the syncude product. However, for the purposes of this disclosure pentane is usually included as part of the syncrude fraction, and it should be assumed to be so in the following discussion unless the context indicates otherwise. In addition, some butane may be included in the syncrude up the vapor pressure specification for the final export product.

Sales gas refers to a C₂ minus fraction, i.e., a fraction composed primarily of methane and ethane. Sales gas may in some instances be exported from the production site to market, or the sales gas may in other instances be burned as fuel, flared, or reinjected.

The term “disproportionation” is used in this disclosure to mean the conversion of alkanes or olefins to new hydrocarbons of both lower and higher molecular weight. For example, the alkane, butane, may be converted by disproportionation according to the following reaction:

2 C₄H₁₀←→C₃H₈+C₅H₁₂

“Alkane” as used in this disclosure refers to a branched or unbranched hydrocarbon molecule which is completely saturated with hydrogen and having the general formula CₙH₂ₙ₊₂. Alkanes, are also commonly referred to as paraffins.

An “olefin” is a branched or unbranched hydrocarbon molecule which is not completely saturated with hydrogen. Olefins have the general formula CₙH₂ₙ. Olefins are important in the present invention because they are believed to serve as an intermediate species in the disproportionation reactions of the alkanes.

The disproportionation of saturated hydrocarbons has been described in the patent literature in U.S. Pat. Nos. 3,484,499; 3,668,268; 3,856,876; 3,864,417; and 3,953,537. In the general literature see Hughes, T. R., et. al., Proc. Int. Congr. Catal., 5th (Paper 87) 1972 and Burnett R. L., et. al., Jour. of Cat. 31, pp 55-64, 1973. In the petroleum industry, disproportionation has been proposed for the conversion of refinery gases (see, for example, U.S. Pat. No. 3,773,845) and for the reforming of distillate transportation fuels (see, for example, U.S. Pat. No. 4,676,885).

The process described in this disclosure is designed to convert the unsaleable gaseous fractions, such as LPG, to higher value products such as syncrude which have greater value on a volumetric basis than the equivalent volume of LPG. The process may be used to convert only part of the unsaleable gaseous fractions, but preferably the process is operated to convert all of the unsaleable fractions to saleable products. An additional advantage of the process of the present invention is that some of the by-products can be mixed with natural gas for transport to market and hence realization of commercial value. Alternately, the by products may be economically disposed of through facilities that already exist for other purposes.

### SUMMARY OF THE INVENTION

In its broadest aspect the present invention is directed to a process for recovering saleable product from the well gas, said process comprising the steps of (a) separating the well gas into an alkane-containing gaseous fraction and a condensate product having a dew point above said gaseous fraction; (b) contacting at least a portion of the gaseous fraction in a disproportionation zone with a disproportionation catalyst under conditions selected to convert a significant portion of the alkanes in said gaseous fraction by disproportionation into both higher and lower alkanes; (c) recovering alkanes from the disproportionation zone; and (c) separating the alkanes into saleable products. Preferably the process will be operated to completely convert all of the gaseous fraction to saleable products. However, in some instances it may not be feasible to completely convert all of the gaseous fraction to saleable product and an amount of unmarketable gas will remain for disposal. This unmarketable gaseous fraction is referred to in this disclosure as light hydrocarbon waste gas. One skilled in the art will recognize that the exact composition of the saleable products and the light hydrocarbon waste gas will vary with the operation and will depend on such factors as the original composition of the well gas, the market into which the products are sold, the specifications for the products, and the transportation costs. Generally, light hydrocarbon waste gas will include LPG. It may also include sales gas if the cost of transporting this fraction to market exceeds its commercial value or the facilities necessary for its transportation are not available.

The process of the present invention is usually operated as a continuous process, and will usually be operated with various recycle loops which recycle at least a portion of the unsaleable alkanes, usually butane and/or propane, recovered from the disproportion zone back into the disproportion zone for further conversion. It should also be understood that the terms “higher alkane” and “lower alkane” as used in this disclosure are relative terms that refer to different hydrocarbon fractions which may be separated by their dew points. Lower alkanes refers to those alkane fractions which contain relatively fewer carbon atoms in the molecule as compared to higher alkanes. As will be explained below, the disproportionation process converts the original alkane molecules into new alkane molecules which have both a larger number of carbon atoms and a smaller number of carbon atoms in their respective molecules. However the average molecular weight of the molecules in the feed and in the products following disproportionation will remain the same.

Any light hydrocarbon waste gas produced by the process may be disposed of in various ways. It may be used locally as a fuel, flared, or reinjected back into the underground formation. The selection of the disposal means will depend on economics and environmental factors. The light hydrocarbon waste gas also may be reinjected into the producing formation for pressure maintenance or as part of a secondary recovery project. In both of these situations, it is for the purpose of improving the recovery of the crude oil and not simply as a means of disposing of the unsaleable gas. When the light hydrocarbon waste gas is reinjected into the ground, it is sometimes referred to as injection gas. When sales gas is recovered as saleable product, the light hydrocarbon waste gas recovered from the disproportionation zone, if there is any, consists primarily of propane, and that portion of the butane which is not included with the syncrude. In some instances some pentane may also be included in the light hydrocarbon waste gas. The amount of pentane and butane that is included in the syncrude product will be dependent on the vapor pressure specification for the final export product. In those instances where the sales gas is not recovered as a saleable product, the light hydrocarbon waste gas also will include methane and ethane.

When the sales gas is disposed of as part of the light hydrocarbon waste gas, the invention may be described as a continuous process for the production of saleable product from the well gas wherein the C₄ minus hydrocarbons from the well gas are partially converted to a C₅ plus product comprising the steps of contacting the C₄ minus hydrocarbons in a disproportionation zone with a disproportionation catalyst under conditions selected to convert a significant portion of the C₄ minus hydrocarbons to a C₅ plus product; separately recovering the C₅ plus product from a light hydrocarbon waste gas consisting of C₄ minus hydrocarbons; and disposing of the light hydrocarbon waste gas. In this instance, substantially all of the pentane fraction is recovered as part of the syncrude fraction. In those instances in which the pentane fraction is not included as part of the syncrude product but remains as part of the light hydrocarbon waste gas, the present invention may be described as a continuous process for the production of saleable product from the well gas wherein the C₅ minus hydrocarbons from the well gas are partially converted to a C₆ plus product which comprises the steps of contacting the C₅ minus hydrocarbons in a disproportionation zone with a disproportionation catalyst under conditions selected to convert a significant portion of the C₅ minus hydrocarbons to a C₆ plus product; separately recovering the C₆ plus product from the light hydrocarbon waste gas which consists primarily of C₅ minus hydrocarbons; and disposing of the light hydrocarbon waste gas.

In those instances in which the sales gas is recovered as saleable product separate from syncrude and any the light hydrocarbon waste gas, the process may be described as a process for converting the LPG to sales gas and syncrude which comprises contacting the LPG in a disproportionation zone with a disproportionation catalyst under conditions selected to convert a significant portion of the LPG to sales gas and syncrude product; recovering a mixture containing syncrude product and sales gas from the disproportionation zone; and separately recovering the sales gas and syncrude product. In this instance, any light hydrocarbon waste gas remaining after conversion will consist primarily of unconverted LPG which may be recycled for further conversion or disposed of. In those cases where the pentane is recovered as part of the syncrude product, the invention may be described as a continuous process for the conversion of LPG comprised of C₃ and C₄ hydrocarbons to a C₂ minus product and a C₅ plus product which comprises contacting the LPG in a disproportionation zone with a disproportionation catalyst under conditions selected to convert a significant portion of the C₃ and C₄ hydrocarbons in the LPG to a C₂ minus product and a C₅ plus product; recovering a mixture containing C₅ plus product and C₂ minus product from the disproportionation zone; and separating the C₂ plus product and C₅ plus product. In those cases where the pentane is not recovered as part of the syncrude product, the invention may be described as a continuous process for the conversion of LPG comprised of C₃, C₄, and C₅ hydrocarbons to a C₂ minus product and a C₆ plus product which comprises contacting the LPG in a disproportionation zone with a disproportionation catalyst under conditions selected to convert a significant portion of the LPG to a C₂ minus product and a C₆ plus product; recovering a mixture containing C₆ plus product and C₂ minus product from the disproportionation zone; and separating the C₂ plus product and C₆ plus product. In this instance the pentane fraction also may be recovered separately as a saleable product.

According to the present invention disproportionation is used to convert the hydrocarbons in the well gas to both heavier products and lighter products which according to the economics may be transported separately to market or blended with the crude oil recovered from the well for export. Any light hydrocarbon waste gas that is not exported is disposed of on site. The present invention has a number of advantages over conventional ways of handling well gas. First, it converts at least part of the well gas into a higher value product on site. The disproportionation reactions are carried out without added hydrogen, so the present invention does not require the installation of hydrogen production facilities or recycle gas compressors to convert the well gas to other products. However, some compressors and a local supply of hydrogen and nitrogen may be necessary for the initial reduction of the catalyst and for catalyst regeneration. This requirement would be periodic and not continuous. The process of the present invention operates at modest pressures. The process of the present invention does not release or consume large amounts of reaction heat, and therefore, it does not require internal control equipment in the reactors to control heat. These factors add up to provide a relatively inexpensive, safe and simple to operate conversion facility for the well gas.

Disproportionation catalysts suitable for carrying out the process of the present invention have been previously described in the literature. The catalyst mass used in carrying out the present invention must have both disproportionation activity and dehydrogenation/hydrogenation activity. Usually the disproportionation activity and dehydrogenation/hydrogenation activity of the catalyst requires separate components for carrying out the different functions, and the catalyst is referred to as a dual function catalyst.

Preferably the disproportionation function will include a metal or mixture of metals selected from Group VIB or Group VIIB of the Periodic Table of the Elements. Particularly preferred for use as disproportionation catalysts are tungsten, rhenium, and molybdenum or the compounds thereof. For the dehydrogenation/hydrogenation function, metals or mixtures of metals and/or the compounds thereof selected from Group VIII of the Periodic Table of the Elements are preferred. Particularly preferred are the noble metals, and most preferably the metal or metal mixture will contain platinum and/or palladium or the compounds thereof. In addition, the presence of rhenium has been found to enhance the activity of the metals used in the dehydrogenation/hydrogenation catalyst.

When used in this disclosure, the Periodic Table of the Elements referred to is the version published by the Chemical Abstracts Service in the Handbook of Chemistry and Physics, 72nd Edition (1991-1992). One skilled in the art will recognize that when referring to the metals which are used as a catalyst for both the disproportionation function and the dehydrogenation/hydrogenation function, the active form of the metal is not necessarily the pure metal. It may be a compound of the metal, such as an oxide of the metal. The specific form of the metal component as it is present during the actual reactions is not known, therefore, when this disclosure refers to a specific metal as acting as a catalyst in a reaction, it should be understood that the exact compound and/or oxidation state of the metal is not known.

Usually the metal components used for the disproportionation function and the dehydrogenation/hydrogenation function will be supported on a solid refractory material, such as, but not necessarily limited to, an oxide such as alumina, zirconia, silica, boria, magnesia, or a mixture of two or more of any of the materials, including zeolites and mesoporous materials such as MCM-41. Mesoporous materials as used herein refers to a molecular sieve having pores of uniform size within the range of from about 20 Angstrom to about 200 Angstrom. Carbon may also be used as support. Preferably the support will be a non-acidic support, i.e., a support having few or no free acid sites. Supports which have free acid sites may be neutralized using the cations of the alkali metals, such as that of lithium, making them more suitable for use as a support.

In those catalysts having the different functions on separate components, i.e., separate disproportionation and dehydrogenation/hydrogenation components, the two components preferably are in close proximity to one another. An example of a dual function catalyst suitable for use in the present invention is a catalyst having a platinum-on-alumina component and tungsten-on-silica component.

### BRIEF DESCRIPTION OF THE DRAWING

FIG. 1 is a schematic process flow diagram illustrating a process for converting LPG in well gas to sales gas and syncrude.

FIG. 2 is a schematic flow diagram illustrating another embodiment of the present invention in which part of the well gas is converted to syncrude and the remaining gaseous fraction is reinjected back into the producing formation.

### DETAILED DESCRIPTION OF THE INVENTION

In the process that is the present invention, the various alkane fractions making up the well gas are converted to both lower and higher molecular weight alkanes. For example, the butane in the well gas is converted in the disproportionation reactor primarily to propane and pentane, although some higher and lower molecular weight alkanes, such as hexane and ethane, will also be produced. The pentane is usually recovered as part of the syncrude fraction while the propane becomes part of the unconverted well gas and may be recycled for further conversion or disposed of as by reinjected into the production formation.

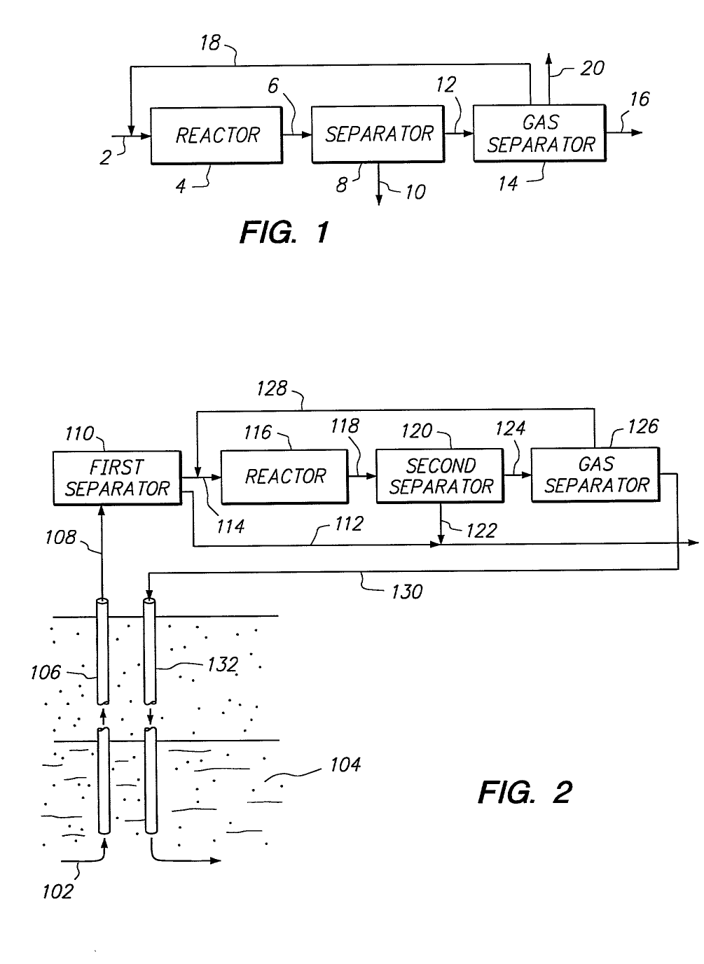

The process of the present invention may be clearly understood by reference to the drawings. FIG. 1 illustrates a continuous process for the conversion of LPG into sales gas and syncrude. A mixture of gases from the well which consist primarily of alkanes having between two and six carbon atoms in the molecular structure are carried by line 2 to the disproportionation reactor 4 where the gases are contacted with a catalyst mass having both dehydrogenation/hydrogenation activity and disproportionation activity. In the reactor the propane in the gas is converted mostly to ethane and butane along with some higher and lower molecular weight alkanes. The butane in the gas is converted to mostly pentane and propane along with some higher and lower molecular weight alkanes. The products are carried from the disproportionation reactor by line 6 to a separator 8 where the C₅ plus fraction is recovered as a liquid through line 10. The C₅ plus fraction is blended with crude oil from the well and is exported to market. The C₂ minus fraction and the unconverted propane/butane are carried by line 12 to a gas separator 14 where the ethane and methane are recovered by line 16. This fraction is exported as sales gas. The propane and butane recovered from the gas separator are recycled by line 18 back to the disproportionation reactor 4 for further conversion. Any excess propane and butane is disposed of through line 20 by means which have been previously discussed.

FIG. 2 illustrates a second embodiment of the invention in which the sales gas fraction is included with the LPG in the light hydrocarbon waste gas and the gases are reinjected back into the producing formation. In this embodiment, a mixture of crude oil and well gas 102 is carried from underground producing formation 104 by production pipe string 106. The oil and gas mixture is carried from the well head by conduit 108 to a first separator 110 where the crude oil product consisting of hydrocarbons having greater than 4 carbon atoms in the molecular structure are separated from the well gas. The well gas is a mixture of gases which consist primarily of alkanes having less than 5 carbon atoms in the molecular structure. The crude oil product is carried by line 112 to storage and eventual export from the production site. The gaseous fraction is carried from the first separator 110 by line 114 to the disproportionation reactor 116 where the gases are contacted with a catalyst mass having both dehydrogenation/hydrogenation activity and disproportionation activity. In the reactor the alkanes in the gaseous fraction are converted to higher and lower molecular weight alkanes. The converted gases are carried from the disproportionation reactor by line 118 to a second separator 120 where the C₅ Plus fraction is recovered as a liquid through line 122. The C₅ Plus fraction in line 122 is blended with crude oil from the well in line 112 and is exported to market along with the crude oil. The C₄ minus fraction may be reinjected as injection gas into the well at this point, or as shown in this embodiment, is carried by line 124 to a gas separator 126 where the butane, propane and any other higher alkanes are recovered from the gas separator and recycled by line 128 back to the disproportionation reactor 116 for further conversion. The lower alkanes, i.e., those alkanes having less than 4 carbon atoms in their molecular structure, are carried by line 130 back to the wellhead and reinjected by means of pipe string 132 back into the underground formation as injection gas.

Depending on its composition, the gaseous fraction may be sent directly to the disproportionation reactor without any prior treatment. However, in most cases some prior treatment may be desirable before the disproportionation step. For example, in the case of those catalysts containing platinum as a dehydrogenation/hydrogenation component, sulfur will act as a moderate poison. In those catalysts which use tungsten or other metals in the VIB or VIIB Groups as a disproportionation component, sulfur would be expected to act as a permanent poison. Therefore, when compounds of sulfur are present in the well gas, it will be preferable to remove this contaminant prior to contact with the disproportionation catalyst. Various methods have been described in the literature which are suitable for the removal of sulfur from the well gas. For example, treatment with amines may be used to remove hydrogen sulfide from the well gas. Organic sulfur compounds, such as mercaptans, may be removed by treatment with caustic or by hydrogenation processes such as hydrotreating. However, in such an instance a local source of hydrogen would be required for the hydrotreating step. Specific commercial processes are available for the removal of sulfur compounds from well gases and are well known to those skilled in the art.

In addition, the presence of ammonia and moisture in the feed to the reactor have been reported to have a deleterious effect on some disproportionation catalysts. Commercial processes that may be used to remove these contaminants from the feed to the disproportionation reactor are well known to those skilled in the art. The presence of excess olefins and hydrogen in the disproportionation zone are also known to effect the equilibrium of the disproportionation reaction and to deactivate the catalyst. Since the composition of the well gas will vary with location, some routine experimentation will be necessary to identify the contaminants that are present and identify the optimal processing scheme and catalyst to use in carrying out the invention.

Various catalysts are known to catalyze the disproportionation reaction. The catalyst mass used to carry out the present invention must have both dehydrogenation/hydrogenation activity and disproportionation activity. The dehydrogenation activity is believed to be necessary to convert the alkanes in the feed to olefins which are believed to be the actual species that undergo disproportionation. Following disproportionation, the olefin is converted back into an alkane. It is theorized that the dehydrogenation/hydrogenation activity of the catalyst also contributes to rehydrogenation of the olefin to an alkane. While it is not intended that the present invention be limited to any particular mechanism, it may be helpful in explaining the choice of catalysts to further discuss the sequence of chemical reactions which are believed to be responsible for disproportionation of the alkanes. As an example, the general sequence of reactions for butane is believed to be:

2C₄H₁₀←→2C₄H₈2H₂←→C₃H₆+C₅H₁₀+2H₂←→C₃H₈+C₅H₁₂

The catalyst mass for use in the disproportionation zone will be dual function and may have the two functions on the same catalyst particle or may consist of different catalysts having separate dehydrogenation/hydrogenation and disproportionation components within the catalyst mass. The dehydrogenation/hydrogenation function within the catalyst mass usually will include a Group VIII metal from the Periodic Table of the Elements which includes iron, cobalt, nickel, palladium, platinum, rhodium, ruthenium, osmium, and iridium. Usually the dehydrogenation/hydrogenation component will include at least one Group VIII noble metal, such as palladium, platinum, rhodium, ruthenium, osmium, iridium, or various combinations thereof. Platinum and palladium or the compounds thereof are preferred for inclusion in the dehydrogenation/hydrogenation component, with platinum or a compound thereof being especially preferred. In addition, the presence of rhenium in combination with the noble metal is desirable. Particularly preferred are catalysts containing a mixture of platinum and rhenium. As noted previously, when referring to a particular metal in this disclosure as being useful in the present invention, the metal may be present as elemental metal or as a compound of the metal. As discussed above, reference to a particular metal in this disclosure is not intended to limit the invention to any particular form of the metal unless the specific name of the compound is given, as in the examples in which specific compounds are named as being used in the preparations.

In the event the catalyst deactivates with the time-on-stream, specific processes which are well known to those skilled in art are available for the regeneration of the catalysts.

Usually the disproportionation component of the catalyst mass will include one or more of a metal or the compound of a metal from Group VIB or Group VIIB of the Periodic Table of the Elements, which include chromium, manganese, molybdenum, rhenium, and tungsten. Preferred for inclusion in the disproportionation component are molybdenum, rhenium, tungsten, and the compounds thereof. Particularly preferred for use in the disproportionation component is tungsten or a compound thereof. As discussed, the metals described, above, may be present as elemental metals or as compounds of the metals, such as, for example, as an oxide of the metal. It is also understood that the metals may be present on the catalyst component either alone or in combination with other metals.

In most cases the metals in the catalyst mass will be supported on a refractory material. Refractory materials suitable for use as a support for the metals include conventional refractory materials used in the manufacture of catalysts for use in the refining industry. Such materials include, but are not necessarily limited to, alumina, zirconia, silica, boria, magnesia, titania and other refractory oxide material or mixtures of two or more of any of the materials. The support may be a naturally occurring material, such as clay, or synthetic materials, such as silica-alumina and borosilicates. Molecular sieves, such as zeolites, also have been used as supports for the metals used in carrying out the dual functions of the catalyst mass. See, for example, U.S. Pat. No. 3,668,268. Mesoporous materials such MCM-41 and MCM-48, such as described in Kresge, C. T., et. al., Nature (Vol. 359) pp. 710-712, 1992, may also be used as a refractory support. Other known refractory supports, such as carbon, may also serve as a support for the active form of the metals in certain embodiments of the present invention. The support is preferably non-acidic, i.e. having few or no free acid sites on the molecule. Free acid sites on the support may be neutralized by means of alkali metal salts, such as those of lithium. Alumina, particularly alumina on which the acid sites have been neutralized by a alkali salt, such as lithium nitrate, is usually preferred as a support for the dehydrogenation/hydrogenation component, and silica is usually preferred as the support for the disproportionation component.

The amount of active metal present on the support may vary, but it must be at least a catalytically active amount, i.e., a sufficient amount to catalyze the desired reaction. In the case of the dehydrogenation/hydrogenation component the active metal content will usually fall within the range from about 0.01 weight percent to about 50 weight percent on an elemental basis, with the range of from about 0.1 weight percent to about 20 weight percent being preferred. For the disproportionation component, the active metals content will usually fall within the range of from about 0.01 weight percent to about 50 weight percent on an elemental basis, with the range of from about 0.1 weight percent to about 15 weight percent being preferred.

A typical disproportionation catalyst for use in the present invention which includes a platinum component and a tungsten component is described in U.S. Pat. No. 3,856,876, the entire disclosure of which is herein incorporated by reference. In one embodiment of the present invention a catalyst is employed which comprises a mixture of platinum-on-alumina and tungsten-on-silica, wherein the volumetric ratio of the platinum component to the tungsten component is greater than 1:50 and less than 50:1. Preferably the volumetric ratio of the platinum component to the tungsten component in this particular embodiment is between 1:10 and 10:1.

Both the dehydrogenation/hydrogenation component and the disproportionation component may be present within the catalyst mass on the same support particle as, for example, a catalyst in which the dehydrogenation/hydrogenation component is dispersed on an unsupported disproportionation component such as tungsten oxide. In another embodiment of the invention, the catalyst components may be separated on different particles. When the dehydrogenation/hydrogenation component and the disproportionation component are on separate particles, it is preferred that the two components be in close proximity to one another, as for example, in a physical mixture of the particles containing the two components. However, in other embodiments of the invention, the components may be physically separated from one another, as for example, in a process in which separate dehydrogenation/hydrogenation and disproportionation zones are present in the reactor. In a reactor having a layered fixed catalyst bed, the two components may, in such an embodiment, be separated in different layers within the bed. In some applications it may even be advantageous to have separate reactors for carrying out the dehydrogenation and disproportionation steps. However, in processing schemes where the dehydrogenation of the alkanes to olefins occurs separately from the disproportionation reaction of the olefins, it may be necessary to include an additional hydrogenation step in the process, since the rehydrogenation of the olefins must take place after the disproportionation step.

The process conditions selected for carrying out the present invention will depend upon the disproportionation catalyst used. In general, the temperature in the reaction zone will be within the range of from about 400 degrees F. (200 degrees C.) to about 1,750 degrees F. (950 degrees C.) with temperatures in the range of from about 500 degrees F. (260 degrees C.) to about 1,350 degrees F. (730 degrees C.) usually being preferred. In general the conversion of the alkanes by disproportionation increases with an increase in pressure. Therefore, the selection of the optimal pressure for carrying out the process will usually be at the highest practical pressure under the circumstances. Accordingly, the pressure in the reaction zone should be maintained above 100 psig, and preferably the pressure should be maintained above 500 psig. The maximum practical pressure for the practice of the invention is about 5000 psig. More typically, the practical operating pressure will below about 3000 psig. The feedstock to the disproportionation reactor should contain a minimum of olefins, and, preferably, should contain no added hydrogen.

Platinum/tungsten catalysts are particularly preferred for carrying out the present invention because the disproportionation reaction will proceed under relatively mild conditions. When using the platinum/tungsten catalysts, the temperature should be maintained within the range of from about 400 degrees F. (200 degrees C.) to about 1200 degrees F. (650 degrees C.), with temperatures above about 500 degrees F. (260 degrees C.) and below about 1000 degrees F. (540 degrees C.) being particularly desirable.

One skilled in the art will recognize that the reactions that occur in the disproportionation zone are equilibrium reactions and, as such, it is desirable to reduce the concentration of the desired products in the disproportionation zone to as low a concentration as possible to favor the reactions in the desired direction. Therefore, it is desirable to remove as much of the C₅ plus hydrocarbons from the well gas prior to its introduction into the disproportionation zone. In addition, it is preferred that the process be carried under conditions selected to minimize the amount of methane produced in the disproportionation zone. As such, some routine experimentation may be necessary to find the optimal conditions for conducting the process.

### EXAMPLE 1

A dehydrogenation/hydrogenation catalyst component was prepared by dissolving 0.3446 grams of Pt(NH₃)₄(NO₃)₂ and 1.7263 grams of LiNO₃ in 49.0 grams of water. The solution was impregnated overnight in 34.4 grams of Catapal alumina (42-60 mesh fraction). The impregnated particles were calcined in air initially at a temperature of 250 degrees F., raised to 1004 degrees F. over a period of 5 hours, and held for 5 hours at 1004 degrees F. The catalyst component was cooled to room temperature within about 5 hours.

### EXAMPLE 2

A disproportionation component was prepared by dissolving 1.9886 grams of ammonium metatungstate (90.6 wt. % WO₃) in 48.0 grams of water. The solution was impregnated overnight on 20.72 grams of silica gel manufactured by W.R. Grace/Davison (silica gel grade 57, 42-60 mesh fraction). The resulting impregnated material was calcined in the same manner as the component described in Example 1, above.

### EXAMPLE 3

The disproportionation catalyst was prepared by mixing 2.25 cc of the dehydrogenation/hydrogenation component prepared in Example 1 and 1.75 cc of the disproportionation component prepared in Example 2. The catalyst mixture (4.0 cc catalyst volume) was loaded into a ¼ inch stainless steel tube reactor which was mounted into an electric furnace containing three heating zones. The catalyst mixture was first dried in nitrogen flow (100 cc/min.) from room temperature to 400 degrees F. within a period of one hour. The mixture was reduced in hydrogen flow (100 cc/min.) using a temperature program consisting of 400 degrees F. to 900 degrees F. within one hour and holding it at 900 degrees F. for 12 hours. Subsequently the catalyst mixture was purged with a nitrogen flow for about one hour and cooled to 800 degrees F. The reactor was pressurized to 900 psig with nitrogen. The nitrogen was switched to a hydrocarbon feed consisting of either n-butane or propane delivered at a rate of 4.0 cc/hr. The results of the disproportionation reactions for n-butane are shown in Table 1 and for propane are shown in Table 2.

TABLE 1

| | |
|---|---|
| N-Butane Conversion, wt. % | 71.8 |
| Yield, wt. % | |
| Methane | 0.4 |
| Ethane | 5.4 |
| Propane | 30.2 |
| Pentanes | 15.1 |
| Hexanes | 8.1 |
| Heptanes | 4.4 |
| Octanes | 2.4 |

TABLE 2

| | |
|---|---|
| Propane Conversion, wt. % | 43.1 |
| Yield, wt. % | |
| Methane | 0.1 |
| Ethane | 16.4 |
| Butane | 17.5 |
| Pentanes | 5.2 |
| Hexanes | 1.5 |

The tables illustrate that about 30 weight percent of the butane feed and about 6 weight percent of the propane feed, respectively, was converted to syncrude under the conditions of the example In addition, about 6 weight percent of the butane feed and about 16.5 percent of the propane feed were converted to sales gas.

What is claimed is:

1. A process for recovering saleable products from well gas, said process comprising the steps of separating the well gas into an alkane-containing gaseous fraction and a condensate product having a dew point above said gaseous fraction; contacting at least a portion of the gaseous fraction in a disproportionation zone with a disproportionation catalyst under conditions selected to convert a significant portion of the alkanes in said gaseous fraction by disproportionation into both higher and lower alkanes; and recovering the alkanes from the disproportionation zone as saleable products.

2. The process of claim 1 which is a continuous process for the production of saleable product from the well gas wherein the C₄ minus hydrocarbons from the well gas are partially converted to a C₅ plus product which comprises contacting the C₄ minus hydrocarbons in the disproportionation zone with the disproportionation catalyst under conditions selected to convert a significant portion of the C₄ minus hydrocarbons to a C₅ plus product; and recovering the C₅ plus product separately from a light hydrocarbon waste gas waste gas consisting primarily of the remaining C₄ minus hydrocarbons.

3. The process of claim 1 which is a continuous process for the production of saleable product from the well gas wherein the C₅ minus hydrocarbons from the well gas are partially converted to a C₆ plus product which comprises the steps of contacting the C₅ minus hydrocarbons in the disproportionation zone with the disproportionation catalyst under conditions selected to convert a significant portion of the C₅ minus hydrocarbons to a C₆ plus product; and recovering the C₆ plus product separately from a light hydrocarbon waste gas which consists primarily of C₅ minus hydrocarbons.

4. The process of claim 1 which is a continuous process that includes the additional steps of recovering at least part of the butane from the disproportionation zone apart from to the saleable products and recycling said butane to the disproportionation zone for further conversion.

5. The process of claim 1 which is a continuous process that includes the additional steps of recovering at least part of the propane from the disproportionation zone apart from to the saleable products and recycling said propane to the disproportionation zone for further conversion.

6. The process of claim 1 wherein a fraction containing higher alkanes having a specified dew point is recovered from the disproportionation zone as saleable product and the lower alkanes are recovered as a light hydrocarbon waste gas.

7. The process of claim 1 wherein a higher alkane fraction having a specified dew point is recovered from the disproportionation zone as saleable product and is mixed with the condensate product.

8. The process of claim 1 wherein a fraction containing lower alkanes having a specified dew point is also separately recovered from the disproportionation zone as saleable product.

9. The process of claim 1 wherein the higher alkane fraction is syncrude and the lower alkane recovered as saleable product is sales gas.

10. The process of claim 1 wherein the disproportionation catalyst is a dual function catalyst having a dehydrogenation/hydrogenation component and a disproportionation component.

11. The process of claim 10 wherein the disproportionation component includes at least one active metal on a refractory support in an amount within the range of from about 0.01 weight percent to about 20 weight percent active metal on an elemental basis and the dehydrogenation/hydrogenation includes at least one active metal on a refractory support in an amount within the range of from about 0.01 weight percent to about 50 weight percent on an elemental basis.

12. The process of claim 11 wherein the active metal in the disproportionation component is within the range of from about 0.1 weight percent to about 15.0 weight percent on an elemental basis and the amount of active metal on the dehydrogenation/hydrogenation is within the range of from about 0.1 to about 20 weight percent on an elemental basis.

13. The process of claim 10 wherein the dehydrogenation/hydrogenation component includes at least one metal or a corresponding metal compound selected form the group consisting of iron, cobalt, nickel, ruthenium, rhodium, palladium, osmium, iridium, and platinum.

14. The process of claim 13 wherein the metal is platinum or palladium or a mixture of platinum and palladium or the compounds thereof.

15. The process of claim 14 wherein the dehydrogenation/hydrogenation component also contains rhenium or a compound of rhenium.

16. The process of claim 10 wherein the disproportionation component includes at least one metal or a corresponding metal compound selected from the group consisting of chromium, manganese, molybdenum, tungsten, and rhenium.

17. The process of claim 16 wherein the metal or corresponding metal compound is tungsten, molybdenum, or rhenium.

18. The process of claim 17 wherein the disproportionation component includes tungsten or a compound thereof.

19. The process of claim 11 wherein the dehydrogenation/hydrogenation component includes platinum or a platinum compound and the disproportionation component includes tungsten or a compound of tungsten.

20. The process of claim 19 wherein the disproportionation catalyst is a mixture of platinum-on-alumina and tungsten oxide-on-silica and the volumetric ratio of the platinum component to the tungsten component is greater than 1:50 and less than 50:1.

21. The process of claim 20 wherein the volumetric ratio of the platinum component to the tungsten component is between 1:10 and 10:1.

22. The process of claim 20 wherein the temperature in the disproportionation zone is maintained within the range of from about 500 degrees F. to about 1000 degrees F.

23. The process of claim 10 wherein the temperature in the disproportionation zone is maintained within the range of from about 400 degrees F. to about 1,750 degrees F.

24. The process of claim 1 wherein the disproportionation catalyst includes an active metal on a refractory support.

23. The process of claim 24 wherein the refractory support is selected from the group comprising alumina, zirconia, silica, boria, magnesia, and titania or mixtures thereof.

24. The process of claim 23 wherein the refractory support is a molecular sieve.

25. The process of claim 24 wherein the refractory support is a mesoporous material.

26. The process of claim 23 wherein the refractory support includes alumina or silica.

27. The process of claim 1 wherein the pressure in the disproportionation zone is maintained within the range of from about 100 psig to 5000 psig.

28. The process of claim 27 wherein the pressure is maintained within the range of about 500 psig to about 3000 psig.

29. A process for recovering saleable product from the well gas produced from an oil and gas well which comprises separating the well gas into a crude oil product having a pre-selected vapor pressure and a gaseous fraction; contacting a portion of the gaseous fraction in a disproportionation zone with a disproportionation catalyst under conditions selected to convert a significant portion of the gaseous fraction to a syncrude product; separately recovering the syncrude product from the remaining light hydrocarbon waste gas; and disposing of the light hydrocarbon waste gas.

30. The process of claim 29 which is a continuous process for the production of saleable product from the well gas wherein the C₄ minus hydrocarbons from the well gas are partially converted to a C₅ plus syncrude product which comprises the steps of contacting the C₄ minus hydrocarbons in a disproportionation zone with a disproportionation catalyst under conditions selected to convert a significant portion of the C₄ minus hydrocarbons to a C₅ plus syncrude product; separately recovering the C₅ plus syncrude product from the remaining C₄ minus hydrocarbons; and disposing of the unconverted C₄ minus hydrocarbons.

31. The process of claim 29 which is a continuous process for the production of saleable product from the well gas wherein the C₅ minus hydrocarbons from the well gas are partially converted to a C₆ plus syncrude product which comprises the steps of contacting the C₅ minus hydrocarbons in a disproportionation zone with a disproportionation catalyst under conditions selected to convert a significant portion of the C₅ minus hydrocarbons to a C₆ plus syncrude product; separately recovering the C₆ plus syncrude product from the remaining C₅ minus hydrocarbons; and disposing of the unconverted C₅ minus hydrocarbons.

32. The process of claim 29 wherein the light hydrocarbon waste gas is reinjected back into the producing formation.

33. A process for converting LPG to sales gas and syncrude which comprises contacting the LPG in a disproportionation zone with a disproportionation catalyst under conditions selected to convert a significant portion of the LPG to sales gas product and syncrude product; recovering a mixture of syncrude product and sales gas product from the disproportionation zone; and separately recovering the sales gas product and syncrude product.

34. The process of claim 33 wherein C₃ and C₄ hydrocarbons in the LPG are converted to a C₂ minus product and a C₅ plus product which comprises contacting the LPG in the disproportionation zone with a disproportionation catalyst under conditions selected to convert a significant portion of the C₃ and C₄ hydrocarbons in the LPG to a C₂ minus product and a C₅ plus product; recovering a mixture of C₅ plus product and C₂ minus product from the disproportionation zone; and
separating the C₂ plus product and C₅ plus product.

35. The process of claim 33 wherein C₃, C₄, and C₅ hydrocarbons are converted to a C₂ minus product and a C₆ plus product which comprises contacting the LPG in a disproportionation zone with a disproportionation catalyst under conditions selected to convert a significant portion of the LPG to a C₂ minus product and a C₆ plus product; recovering a mixture of C₆ plus product and C₂ minus product from the disproportionation zone; and separating the C₂ plus product and C₆ plus product.

36. The process of claim 33 wherein unconverted LPG is also recovered from the disproportionation zone.

37. The process of claim 36 wherein the LPG recovered from the disproportionation zone is recycled back to the disproportion zone for further conversion.

38. The process of claim 37 wherein substantially all of the LPG is converted to saleable products.

39. The process of claim 33 wherein the pressure in the disproportionation zone is maintained within the range of from about 500 psig to about 3000 psig.

40. The process of claim 33 wherein the process conditions are preselected to minimize the production of methane in the disproportionation zone.
